(19) United States
(12) Patent Application Publication
(10) Pub. No.: US 2005/0214936 A1
(43) Pub. Date: Sep. 29, 2005

(54) Enzyme facilitated solubilization of carbon dioxide from emission streams in novel attachable reactors/devices

(76) Inventor: Sanjoy K. Bhattacharya, Cleveland Heights, OH (US)

Correspondence Address: SANJOY K. BHATTACHARYA, 1555 WOOD ROAD, CLEVELAND HEIGHTS, OH, 44121, US

(21) Appl. No.: 11/010,940

(22) Filed: Dec. 13, 2004

Related U.S. Application Data
(63) Continuation of application No. 10/464,789, filed on Jun. 18, 2003, now abandoned.

(51) Int. Cl. C12M 1/14

(52) U.S. Cl. 435/299.1; 435/266

(57) ABSTRACT

This invention pertains to novel reactors/devices (shall hitherto be named as component/module O) that employs immobilized biocatalysts enabling concentration and solubilization of emitted CO₂ by allowing catalytic contacting with water spray. These novel reactors or devices (module O) will be coupled to module A of devices (Module A, B and C) described in U.S. Pat. No. 6,258,335 resulting in fixation of biochemical conversion of the carbon dioxide on to acceptor RuBP.

### BACKGROUND OF THE INVENTION

Anthropogenic carbon dioxide emission has severe impact on climate. It is regarded as a global pollution problem and has been implicated in global warming (Joos et. al., 1999; Schnur, 2002). Controlling carbon dioxide pollution can be best achieved if abatement is attempted at source. The fixation of carbon in the carbon dioxide in concatenated form in compounds is the best way of holding the carbon in the fixed state for a long term compared to one step terminal fixation such as in the form of carbonate by combining with metal oxides (Bhattacharya, 2001; Bhattacharya et. al., 2002). Biochemical fixation of carbon dioxide readily renders fixation in concatenated forms, although chemical fixation of carbon dioxide into concatenated carbon compounds is also possible. However, any biochemical (or chemical) fixation of carbon dioxide from emission sources would need a concentration step. Biotechnological solution to fixation would necessitate an aqueous solubilization step in addition to concentration before the carbon dioxide is provided for biocatalytic fixation into concatenated carbon compounds. A recyclable bioprocess enabling continuous fixation was invented for the abatement of carbon dioxide pollution at source (Bhattacharya, 2001; Bhattacharya et. al., 2002). This device was built based on a modular approach (comprised of module A, B and C), in one module the carbon dioxide is fixed on 5-carbon acceptor RuBP using Rubisco, named module A (Bhattacharya, 2001; Chakrabarti et. al., 2003a, b) and in other module using a cohort of enzymes the RuBP is regenerated from 3-phosphoglycerate (3-PGA) named module C. Energy for driving the recycling is derived from solar radiation (Bhattacharya, 2001), although, any other form of energy can also be used to propel the RuBP regeneration.

The capture of carbon dioxide from emission stream and the maintenance of the concentration of carbon dioxide near the active site of Rubisco in the immobilized bioreactor are two great challenges. It is widely believed that attempts to capture the carbon dioxide from the emission stream would be associated with a decrease in pressure in the outlet (pressure-drop) leading to an increase in pressure (back-pressure) in the inlet part of the emission stream. A process for direct capture of carbon dioxide from emission of the exhaust/stream for concentration and solubilization is lacking. Thus any device using employing a biotechnological process for concentration and solubilization of carbon dioxide directly from emission steam for further biochemical (or chemical) conversion of the later do not exist. Direct capture of carbon dioxide from emission stream solely using immobilized Rubisco limits the capture rate. This led to the construction of the present novel trickling spray reactor employing immobilized carbonic anhydrase that enables concentration of carbon dioxide from emission stream without generating the back-pressure for the emission stream. Carbonic anhydrase is one of the fastest enzymes that make faster mass transfer from gas phase to aqueous phase, which may then be fed to coupled-Rubisco reactors enabling effective conversion of captured carbon dioxide. The immobilized carbonic anhydrase would make the fast capture and render the gas to be fast solubilized and also prevent the escape of carbon dioxide. The coupled multiple immobilized reactors will allow controlled release of soluble carbon dioxide near the active site of Rubisco and therefore conversion of the captured carbon dioxide into fixed or concatenated state. The notion that carbon dioxide pollution can be abated by fixation at source has been continuously discounted in scientific literature (Beckmann, 1999) and this has stifled research and development in this area that includes processes and devices for the concentration and solubilization of carbon dioxide.

Carbonic anhydrase (CA, EC 4.2.1.1), a zinc metalloenzyme catalyzes the reversible hydration of CO₂ and the dehydration of HCO₃⁻ and plays a significant role in processes such as pH homeostasis, respiratory gas exchange, photosynthesis and ion transport (Badger and Price, 1994; Coleman, 1991; Tashian, 1989). It is widely distributed in tissues of plants and animals (Badger and Price, 1994; Sultemeyer et. al., 1993; Maren, 1967; Maren and Sanyal, 1983), in several members of archea (Karrasch et. al., 1989), in cyanobacteria (Ingle and Coleman, 1975; Kaplan et. al., 1990) and in a variety of eubacteria (Maren and Sanyal, 1983; Suzuki et. al., 1994). The CA can be divided into three major groups based on amino acid sequence, (a) the α, or eukaryotic group, which includes CA found in vertebrates; (b) the β or bacterial group which includes CA enzymes in eubacteria and similar isoforms in higher-plant chloroplast and cytosol and a group γ, or archaebacterial group of CA which plays a role in acetate metabolism (Holmes, 1977; Karrasch et. al., 1989; Alber and Ferry, 1994; Hewett-Emmett and Tashian, 1996). CA plays an important role in photosynthesis and in the operation of the CO₂-concentrating mechanism (CCM) in achaebacteria, eubacteria and cyanobacteria (Kaplan et. al., 1990). Efficient photosynthetic inorganic carbon (Ci) assimilation by cyanobacteria, archaebacteria and in some eubacteria at limiting available levels of Ci necessitates operation of CCM. As a process, CCM increases the CO₂ concentration around primary carboxylating enzyme, Ribulose-1,5-bisphosphate carboxylase/oxygenase (Rubisco) to levels several orders of magnitude above that present in the surrounding medium enabling enhancement of rate of photosynthesis (Badger and Price, 1994; Kaplan et. al., 1990; Miller et. al., 1990). The intracellular CO₂ concentration is elevated as a result of a two-step process both steps involving CA activity. First, light-dependent inorganic carbon transport systems, which utilize both CO₂ and HCO₃⁻ activity accumulate Ci in the cytosol. Second, the accumulated Ci, which is present mostly in the form of HCO₃⁻ is dehydrated to CO₂, the actual substrate of Rubisco. The bicarbonate dehydration, catalyzed by CA, occurs close to the active site of Rubisco (Price et. al., 1992).

The capture of carbon dioxide from emission streams in enzymatic aqueous trapping and the ability to deliver concentrated carbon dioxide at the site of catalytic conversion or fixation step that would hold/convert carbon dioxide in fixed concatenated state such as at the active site of Rubisco holds major scope for development (Bhattacharya, 2001). The bioprocess for fixation of Rubisco has been previously employed using highly enriched stream of carbon dioxide (Bhattacharya, 2001) after preliminary treatment of stream from emission sources. The mass transfer rate of carbon dioxide from gas phase to aqueous phase is one of the rate limiting steps. The residence time of CO₂ in the aqueous phase determines the fate of its being fixed by Rubisco in the biocatalytic fixation chamber. Additionally the forced mass transfer of gas across liquid phase (solution of Rubisco and RuBP) results in building back-pressure in the emission stream. Facilitated enzyme assisted mass transfer is expected to enhance solubility of CO₂ in gas phase making it available for conversion by Rubisco and help reduce the back-pressure in the emission stream. A number of different methods have been used for enzyme immobilization including carbonic anhydrase (Manecke and Schlunsen, 1976; Turkova, 1976; Manecke and Vogt, 1980; Salley et. al., 1992; Gagnon et. al., 1994; Azari and Nemat-Gorgani, 1999; Liu et. al., 2001; Simsek-Ege et. al., 2002; Bhattacharya et. al., 2003). However, immobilized carbonic anhydrase or any other biocatalyst or chemical agent has not been applied for the concentration and solubilization of carbon dioxide from emission streams. In this patent application immobilized carbonic anhydrase (CA), has been used. CA was immobilized in different porous matrices and water spray instead of solution phase was applied to enhance solubility of CO₂ and hence enhanced capture without any significant pressure drop or back-pressure in the emission stream with facilitated mass transfer to aqueous phase. At the same time possibility of feeding captured solubilized carbon dioxide for biochemical (or chemical) conversion such as using an immobilized Rubisco reactors (module A in U.S. Pat. No. 6,258,335) exists with this invention to help enhance the fixation of captured carbon dioxide. The immobilized carbonic anhydrase has been used in a novel way using trickling spray bioreactors. Such a process has never been applied before makes it novel. The fact that such a process in a novel device allowing simultaneous gas and water flow leading to concentration and solubilization of the carbon dioxide from emission streams makes the process and the devices build along these lines great utility to add one step in abatement of carbon dioxide pollution making the concentrated, solubilized carbon dioxide amenable to biocatalytic fixation.

### BRIEF SUMMARY OF THE INVENTION

A novel biotechnological process where the carbon dioxide in emission is solubilized by contacting with spray water trickling through the immobilized carbonic anhydrase (CA) column (hitherto will be named as module O). The immobilized CA catalyzes solubilization and concentration of the carbon dioxide in the emission stream. The process takes place in a trickling spray reactor/device employing immobilized carbonic anhydrase which is also a novel device have been used and designed for the first time for this purpose. The reactor enables solubilization of carbon dioxide from emission exhausts and allows feeding the solubilized carbon dioxide to coupled-immobilized Rubisco reactors.

The tricking spray employed immobilized CA remains moist and active as a result of constant water spray. The carbonic anhydrase enzyme immobilized on glass or polystyrene coated porous steel (DCC and carboxyl coupling of enzyme) was used (Bhattacharya, et. al. 2003). The design of reactor provides ability to control two different flows, that of emission gases and that of water spray. In the design that has been developed, with respect to flow of gases it was either horizontal inflow and horizontal outflow or vertical inflow and horizontal outflow (or vice versa). With respect to water spray it was either vertical or horizontal. Therefore basic design of the reactor were reduced to three different types (a) with horizontal inflow and outflow of gas and vertical water spray, (b) vertical inflow, horizontal outflow of gas (or vice versa) and vertical water spray and (c) vertical inflow, horizontal outflow of gas (or vice versa) and horizontal water spray (FIGS. 1 & 2A, B, C). The designs that employed vertical inflow of gas, allowed the inflow only from the top but never from the bottom. This is due to stability of matrix in presence of vertical inflow from the bottom.

Carbonic anhydrase enables concentration of CO₂ resulting in formation of bicarbonate that could be fed to a biochemical/chemical catalyst such as Rubisco in a coupled reactor. The fast solubilization of CO₂ catalyzed by immobilized CA helps enhance the mass transfer of CO₂ from gas phase into aqueous phase. Utilizing the porous matrix and water spray is unique as the emission stream does not have to pass through a water column. This would have been the situation had soluble carbonic anhydrase was used. The immobilized CA and constant water spray retains the enzyme activity but offers negligible resistance to emission stream compared to a water column that would involve if soluble immobilized enzyme were used in solution state. This device is unique that it does not impede mass transfer of carbon dioxide from the gas to the aqueous phase and at the same time does not lead to a significant back-pressure in the emission stream. The device employing this process is expected to aid and greatly enhance the biocatalytic fixation of carbon dioxide using coupled bioprocesses involving Rubisco.

### BRIEF DESCRIPTION OF THE SEVERAL VIEWS OF THE DRAWINGS

Figures:

FIG. 1: Design of the concentrator reactor. There were three basic designs of the reactor (A) with horizontal inflow and outflow of gas and vertical water spray, (B) vertical inflow, horizontal outflow of gas (or vice versa) and vertical water spray and (C) vertical inflow, horizontal outflow of gas (or vice versa) and horizontal water spray. The parts are 1. Inlet nozzle for gas/emission, 2. outer lid, 3. water inlet, 4. Sprayer mesh, 5. The main vessel/reactor, 6. The large wire container for holding immobilized enzyme core, 7. Immobilized carbonic anhydrase core, 8. outlet nozzle for gas/emission, 9. The bottom wire mesh for percolation of solution, 10. The holder stand, 11. water outlet, 12. bottom solution holding chamber.

FIG. 2: Sectional view of the device without any marking parenthesis (view for the official gazette). The reactors are: (A) with horizontal inflow and outflow of gas and vertical water spray, (B) vertical inflow, horizontal outflow of gas (or vice versa) and vertical water spray and (C) vertical inflow, horizontal outflow of gas (or vice versa) and horizontal water spray. The parts are identified with identical numbers as in FIG. 1: 1. Inlet nozzle for gas/emission, 2. outer lid, 3. water inlet, 5. The main vessel/reactor, 8. outlet nozzle for gas/emission, 11. water outlet, 12. bottom solution holding chamber.

FIG. 3: Percent CO₂ reduction with varying flow rate and varying gas composition in the emission. These measurements were done using a simulated stack type emission, where parameters can be varied unlike actual emission. The enzyme load of 1.5 mg/ml using a gas composition of 33-40 percent was made for flow rate studies. An emission flow rate of 4-5 L/min was used for gas composition studies. The measurements for both flow rate and gas composition were made (analyzed) per liter of solution used for extraction. The symbol (⋄) and (□) indicates flow rate and gas composition respectively. The gas carbon dioxide composition with and without reactor was measured (Testo 400 multifunction equipped with 06321240 CO₂ probe) and this was correlated with the pH based CA activity measurements (Bhattacharya et. al., 2003).

FIG. 4: Percent CO₂ reduction with varying the ratio of water spray area to core immobilized CA volume [Length (L)/Diameter (D) ratio]. The immobilized core had a constant volume of 100 ml for these measurements, the enzyme load of 1.5 mg/ml was used and the gas composition was about 40 percent in the simulated stack type emission. The gas carbon dioxide composition was measured with and without attachment of the immobilized CA reactor.

FIG. 5: Percent CO₂ reduction with varying water flow rate. The water flow rate was varied between 1-12.5 ml/min. The enzyme load of 1.5 mg/ml and average gas flow rate of 4-5 L/min with 40 percent CO₂ in the emission stream was used for these measurements. The percent of CO₂ in the emission gas with and without reactor attachment was measured. For correlation the increase in pH was measured in every one liter extracted solution.

FIG. 6: Percent CO₂ reduction with variation in enzyme load in the matrix of immobilization. The enzyme load was varied between 0.25-10 mg/ml with a constant core volume of 100 ml and average gas flow rate of 4-5 L/min with 40 percent CO₂ in the emission stream was used for these measurements. The percent of CO₂ in the emission gas with and without reactor attachment was measured. For correlation the increase in pH was measured in every one liter extracted solution.

FIG. 7: Percent CO₂ reduction with variation in immobilization matrix pore size. For these measurements the enzyme load was kept 1.5 mg/ml with a constant core volume of 100 ml and the average matrix pore size were selected between 0.5-5 μm. The average gas flow rate of 4-5 L/min with 40 percent CO₂ in the emission stream was used for these measurements. The percent of CO₂ in the emission gas with and without reactor attachment was measured. For correlation the increase in pH was measured in every one liter extracted solution.

FIG. 8: The determination of pressure drop as a result of attachment of a module in the emission stream. The effect of varying reactor diameter on pressure drop in the outlet and the back-pressure in the inlet. The reactor diameter is the barrier that emission stream has to traverse and this was varied between 100 cm to 1000 cm. For these measurements the enzyme load was kept 1.5. mg/ml, the average gas flow rate of 4-5 L/min with 40 percent CO₂ in the emission stream was used, (□) outlet pressure and (▪) inlet pressure respectively. The percent of CO₂ in the emission gas with and without reactor attachment was measured. For correlation the increase in pH was measured in every one liter extracted solution.

FIG. 9: Determination of pressure drop in the outlet and the back-pressure in the inlet with varying matrix pore diameter. Using a core immobilized matrix diameter of 100 cm the matrix pore diameter was varied between 0.5 to 5 μm, all other parameters were same as described in FIG. 8, (□) outlet pressure and (▪) inlet pressure respectively. The percent of CO₂ in the emission gas with and without reactor attachment was measured using Testo 400 multifunction instrument equipped with 06321240 CO₂ probe (Hotek Technologies, Tacoma, Wash.).

FIG. 10: A comparison of CO₂ reduction using multiple reactors and a single reactor with comparable volume of combined multiple reactors. These reactors had an enzyme load of about 0.5 mg/ml. It is illustrated that the single reactor (core volume 1000 ml) had the combined volume of four small reactors (core volume 250 ml) used for CO₂ solubilization/extraction from emission stream. The percent of CO₂ in the emission gas with and without reactor attachment was measured using Testo 400 multifunction instrument equipped with 06321240 CO₂ probe (Hotek Technologies, Tacoma, Wash.) and was also correlated with the pH based measurement of CO₂ solubilization of aqueous phase per 100 ml solution extracted. (A). The percent CO₂ reduction represented by the bars with horizontal strips and (B). The reactor volumes represented by the bars with vertical strips.

### DETAILED DESCRIPTION OF THE INVENTION:

This invention pertains to a biotechnological process or method whereby the carbon dioxide present in the emission stream (free of soot) could be contacted with water in the presence of immobilized carbonic anhydrase resulting in catalytic solubilization of carbon dioxide in water. The enzyme, carbonic anhydrase is immobilized on glass, polystyrene or silica coated steel matrix using DCC or carboxyl coupling described elsewhere (Bhattacharya, et. al., 2003). The contacting process of gas with water also results in the concentration of CO₂ from emission stream in the aqueous phase. The process operation and measurement methods are described in further detail below after a brief physical description of the reactor(s).

### DESCRIPTION OF THE REACTOR(S).

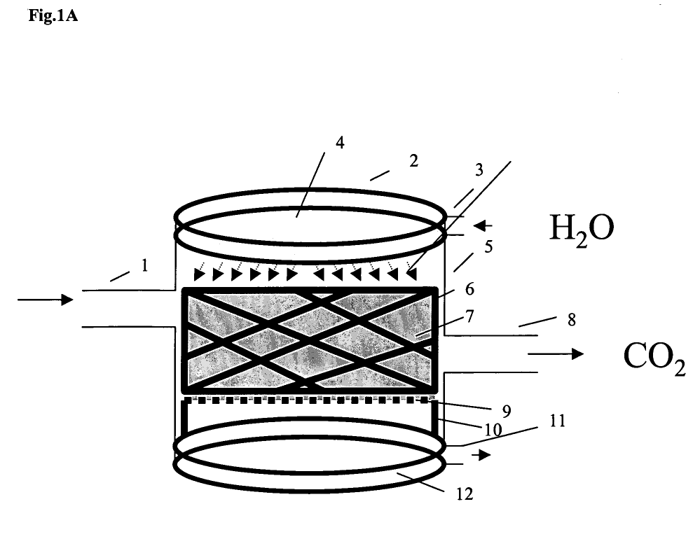

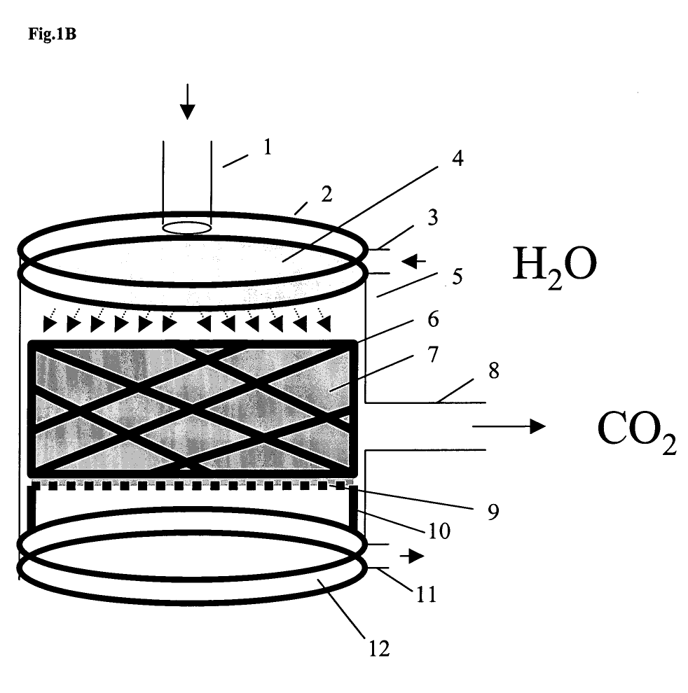

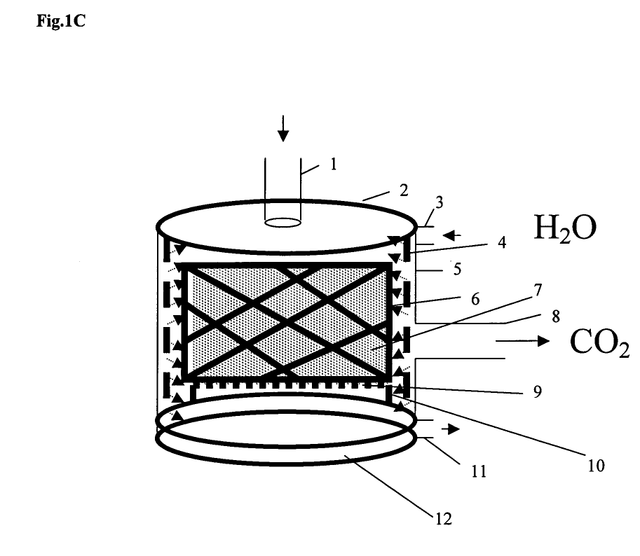

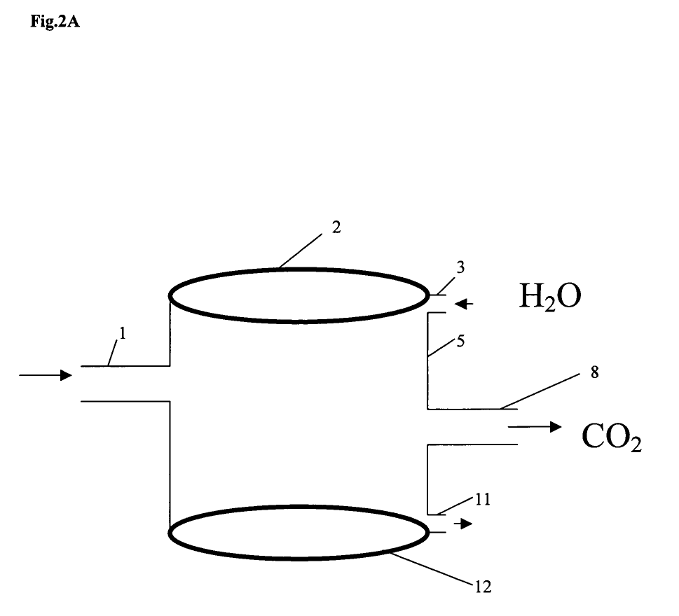

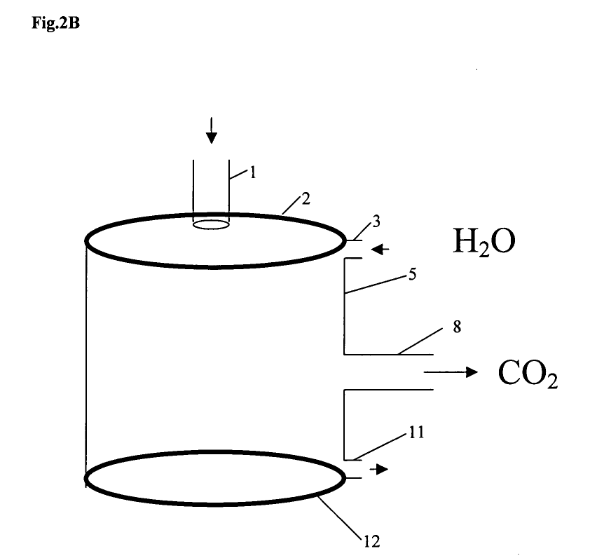

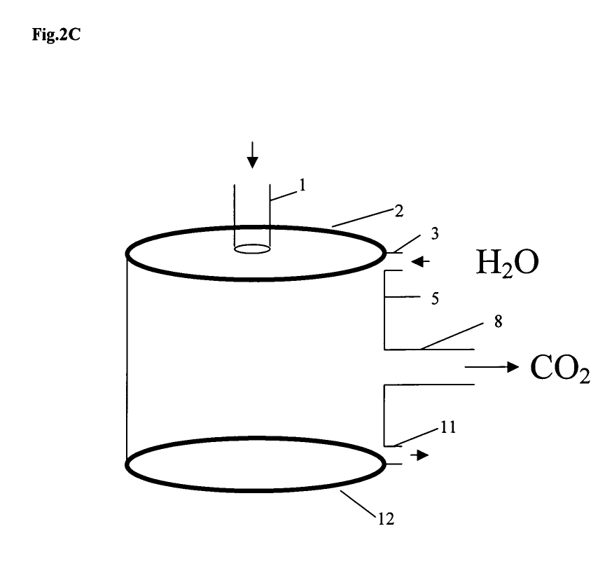

The biotechnological process described above occurs in a novel immobilized carbonic anhydrase (CA) reactor that has been designed and is also an integral part of this invention. This reactor(s) has the highly porous immobilized CA within its core and allows flow of emission gases and water in the form of spray. The reactor parts described in this section pertain to FIGS. 1 and 2. The gas in the reactor enters through a tube connected directly to the emission stream (part 1 in FIGS. 1 and 2). [Not shown here, the highly porous filter offering negligible resistance that holds macroscopic soot and countercurrent water flow across the connector tube bringing the emission gas to the reactor, the treatments needed prior to actual emission stream entry to the reactor cores. The prior treatment renders emission gas free of soot and brings the temperature between 60-80° C. suitable for operation of this biotechnological process, and not being claimed as part of the invention]. The top of the reactor has a lid (part 2), connected with water entry port (part 3) and the bottom of this top portion has a porous lid that allows water spray (part 4). The novel trickling spray reactor has a solid body (part 5), which houses the central immobilized matrix shell (part 7). The shell is encased in a wire mesh (part 6) and sits on a perforated metal plated coated with glass (part 9). The pores in the metal plate (part 9) are 2-5 mm in diameter sits on a strand (part 10) and does not offer mass transfer or flow resistance to aqueous solution/suspension that flows through it. The reactor has one entry port (part 1) and one exit port (part 8) for the flow of emission gas/stream. The emission entry port is either vertical entering from the top or from the horizontal side (FIGS. 1, 2). The reactor also has water inlet (part 3), spray mechanism (part 4) and water/solution outlet (part 11). The bottom of the reactor (part 12) usually collects the aqueous flow and through a single tubing exit (part 11). This solution exit (part 11) could easily be connected with a coupled immobilized Rubisco reactor. The dimension of the cylindrical central part of the reactor is 50 cm×30 cm (diameter×length). The diameter of the gas inflow and outflow tube is 10 cm. However, these dimensions can vary according to the emission stream and other parameters. The 5 cm from the top of this cylindrical central reactor houses water for spray. The water inlet (part 3) and solution outlet (part 11) has a diameter of 2 cm. The spray is governed by lid having pores of diameter 0.5 mm (part 4). The wire mesh encasing (part 6) for immobilized enzyme is made up of steel material having pores with diameter of 5-8 cm. The steel is coated with glass to withstand corrosion.

Reactor Operation and Stability of the Immobilized Biocatalyst.

The reactor described above houses the immobilized enzyme core. The carbonic anhydrase from thermophilic Methanobacterium thermoautotrophicum was cloned in pET19b vector using standard molecular biology protocols as described elsewhere (Smith and Ferry, 1999) was used in the reactor core. Some experiments were also performed using previously reported cloned human carbonic anhydrase IV in pET11d (Waheed et. al., 1997). The enzymes were immobilized on glass, polystyrene or silica coated steel matrix of different average mesh size using methods as reported earlier (Bhattacharya et. al., 2003). The novelty of this biotechnological process lies is using the immobilized enzyme in porous matrix and using water spray instead of solution phase enzyme so that mass transfer resistance to the emission gas is negligible. A thin film of water around the enzyme in the immobilized microenvironment keeps the enzyme hydrated and active for a long time and the buffering of the enzyme apparently is not necessary for a long period. A flush with buffer every third day of continuous operation greatly enhances the shelf-life of the immobilized enzyme. The reactor design, which is the other novel part of this invention, has three basic designs. The reactors in all three designs provide ability to control two different flows, flow of emission gas and that of water spray, with respect to flow of gases it is either horizontal inflow and horizontal outflow or vertical inflow and horizontal outflow (or vice versa). With respect to water spray it was either vertical or horizontal. Therefore basic design of the reactor were reduced to three different types (a) with horizontal inflow and outflow of gas and vertical water spray, (b) vertical inflow, horizontal outflow of gas (or vice versa) and vertical water spray and (c) vertical inflow, horizontal outflow of gas (or vice versa) and horizontal water spray (FIGS. 1 & 2A, B, C). The designs that employed vertical inflow of gas, allowed the inflow only from the top but never from the bottom. This is due to stability of matrix in presence of vertical inflow from the bottom and also the bottom gas inflow would lead to water spray going to the emission stream at least in some design settings. This process and reactor(s) allowing the catalytic contacting of carbon dioxide with water would enable concentration and solubilization and feeding the solubilized CO₂ into coupled fixation bioreactors (Bhattacharya, 2001) and is expected to serve as a great utility. While the prior art exists on enzyme immobilization but there is absolutely no description of contacting carbon dioxide (or emission gas) with water in presence of porous immobilized carbonic anhydrase or anything similar as described in this biotechnological process in printed literature or electronic resources makes this a novel utility. In all these studies simulated stack emission was used generated using a mixture of gases and carbon dioxide derived from dry ice. However, we envisage, based on the operation studies that the device/reactors will work with different emissions including stack emissions. The method using in construction of the device or in measurements are described in the experimental protocol section. The reactor operation optimization studies with respect to different parameters are described below.

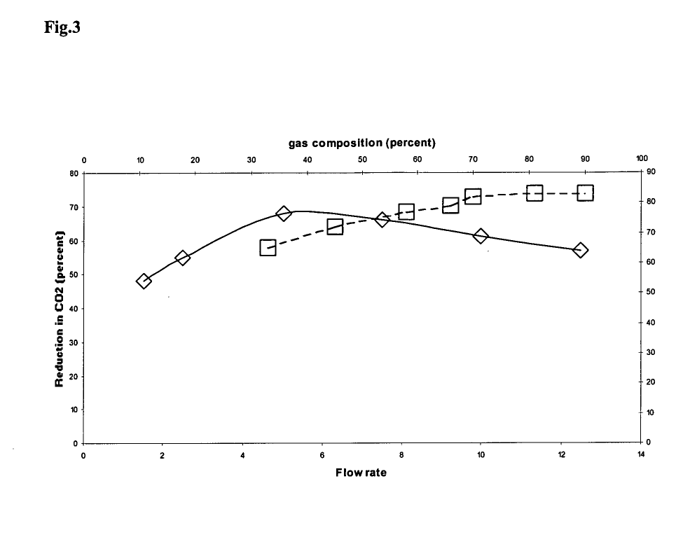

Effect of emission flow rates and CO₂ content in the emission gas on CO₂ reduction. The simulated emission stream where CO₂ percent in the stream was manipulated using gas from dry ice with varying flow rates (having carbon dioxide accounting for about 33-40 percent of the stream) was subjected to treatment using an enzymatic core having an average enzyme load of 1.5 mg/ml. The water flow rate was held constant at 2.5 ml/min mean matrix pore size was 1 μm. As shown in FIG. 3, the reduction in CO₂ initially increased reaching a plateau between 5-7 L/min and the decreases progressively. At each point the CO₂ in the stream without any treatment (without attachment of the reactor core) was treated as 100 percent, based on which a decrease in CO₂ was calculated. The reduction in CO₂ was also measured using an artificially enriched stream of CO₂. At a flow of 4.5 L/min with immobilized enzyme load of 1.5 mg/ml there was a progressive increase in the reduction of CO₂ in the emission stream, which reached a plateau when the carbon dioxide concentration in the emission stream reached around 70 percent (FIG. 3).

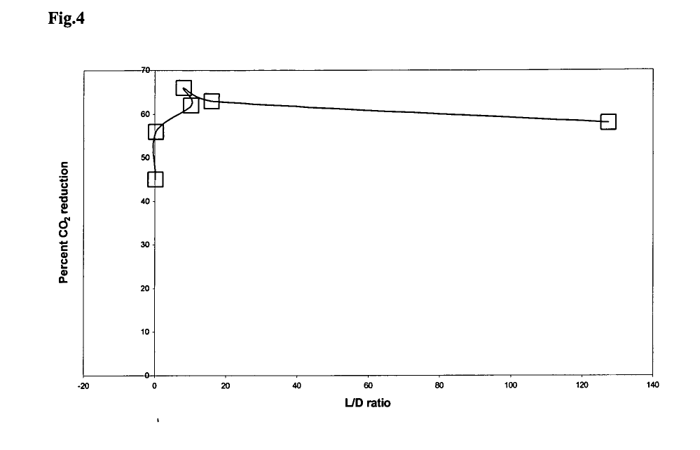

Effect of spray area versus immobilized core volume on CO₂ reduction. The area of spray with respect to core immobilized CA volume affected percent CO₂ reduction, when this biotechnological process was used. In order to understand the effect of spray area to the volume of immobilized enzyme core, the diameter of the core was varied while keeping the volume constant (100 ml). The resultant L/D ratio was calculated and percent CO₂ reduced was determined, where L refers to length and D refers to diameter of the core (FIG. 1). As shown in FIG. 4 the L/D ratio had an effect on CO₂ reduction the either extreme of L/D ratio led to a decrease in reduction. The higher length reduced the mass transfer where as the lower length led to decrease solubilization and rapid escape of carbon dioxide from the immobilized CA core. The intermediate L/D ratio was optimal for proper mass transfer to the active site of CA and hold up of the gas within the immobilized core.

Effect of flow rate of water (spray) on CO₂ reduction. The rate of water flow due to the spray also affected the catalytic solubilization of CO₂. Fast flow of water enabled a constant hydration and availed sufficient water near the active site for catalytic conversion. Flow rate of water was varied from 1 ml/min to 12.5 ml/min. The increase in water flow rate showed an initial increase in the rate of CO₂ reduction and reached a plateau around 8 ml/min (FIG. 5). The availability of water around the immobilized CA affected the CO₂ reduction, which is manifested by increase in reduction with increased flow rate. However, after the flow rate passes limiting rate any further increase in water does not allow further availability of reacting aqueous phase near the active site of enzyme thereby the rate remains unaffected.

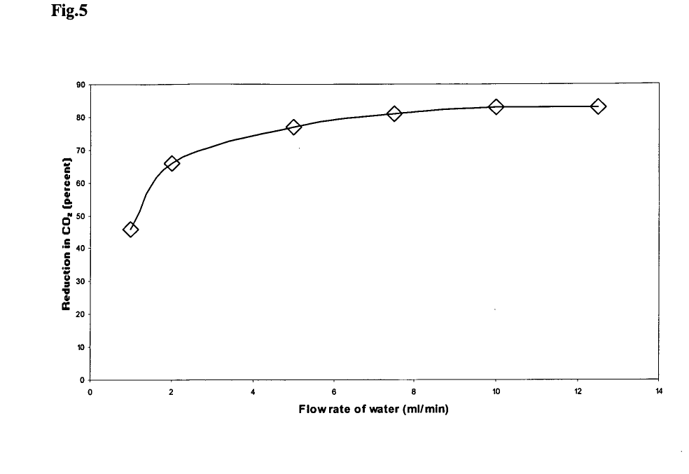

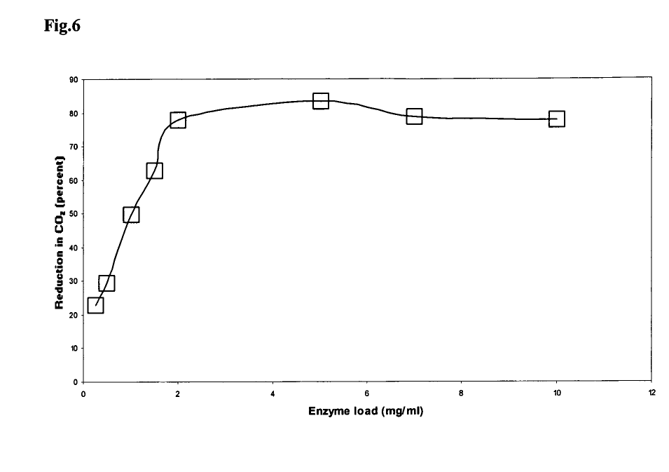

Effect of enzyme load on CO₂ reduction. Immobilized enzyme load had a profound effect on reduction of carbon dioxide. The enzyme load was varied from 0.25 to 10 mg/ml. There was a progressive increase in CO₂ reduction upto 5 mg/ml of enzyme load and beyond this there was a decrease in the CO₂ reduction from the emission stream. The decrease is perhaps due to denaturation of enzyme as well as mass transfer limitation in the enzyme microenvironment with high protein load (FIG. 6).

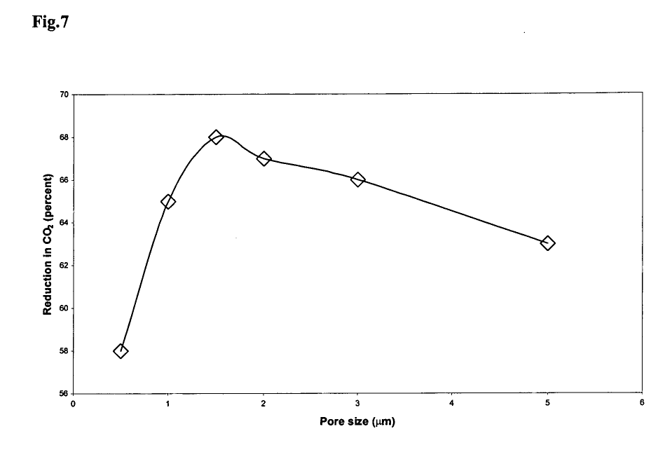

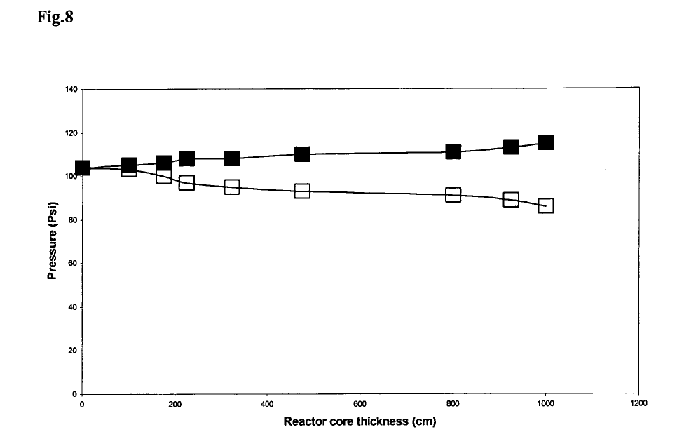

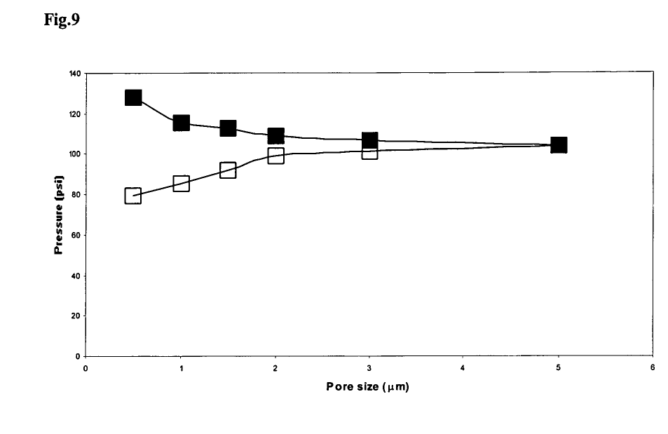

Effect of Immobilized matrix pore size on CO₂ reduction. The matrix pore size influences CO₂ reduction. The average matrix pore size, varied between 0.5 to 5 μm, was determined by mercury intrusion porosimetry utilizing an Aminco-Winslow Porosimeter (Messing, 1970; Messing, 1974) described in experimental protocols. The increase in pore size increases the reduction but beyond a definite size (2 μm) further increase in pore size actually reduces the CO₂ reduction (FIG. 7). The increase in CO₂ reduction with increased pore size is due to increase mass transfer of CO₂ near the active site of immobilized carbonic anhydrase. The observed decrease in CO₂ reduction with large pore size is perhaps due to escape of carbon dioxide from reaching to actual active site of the enzyme immobilized in such matrix. Also the availability of water and diffused carbon dioxide at the same rate in the large pore size matrix may affect the rate of CO₂ reduction. The attachment of the reactor module in the emission stream and pressure drop across the stream. The attachment of the reactor is expected to bring a change in the pressure of outlet (after the reactor) and inlet (before the reactor) within the gas emission. In order to test this, the emission gas pressure before entry to the reactor and at the exit port of the reactor was determined with respect to thickness of reactor core and with varying matix pores using HD8804 K pressure and temperature kit equipped with appropriate pressure probes and also using Testo 525 instrument (Hotek Technologies, Tacoma Wash.). The reactor inlet stream without any reactor connection maintained at a pressure of about 104 Pa. However, we have also used a very high-pressure simulated system for these investigation (data not shown), where we have observed insignificant pressure changes due to attachment of reactors. Using reactor cores of varying diameter (100 to 1000 cm; FIG. 8) as well as matrix pores of 0.5 to 5 μm pressure was measured in the reactor inlet and outlet (FIG. 9). The maximum pressure drop was only 17 percent for more than for an immobilized reactor core with diameter of 1000 cm. The pressure drop in the outlet or back-pressure (that is, pressure increase in the inlet) in the inlet was less than 11 percent till 500 cm core diameter. A commensurate but insignificant increase in inlet pressure (back pressure) was also observed when immobilized reactor core was added (FIG. 8). Using a reactor vessel without an immobilized core water flow alone did not show a significant effect on inlet or outlet pressures (data not shown). The matrix pore size also had an effect on pressure. However the pressure drop with 0.5 μm matrix pore was only about 10.5 percent than without any reactor core control (FIG. 9). The average matrix pore size of 2 μm offered only 5 percent decrease in pressure in the outlet. Decrease in pore size led to increased drop in pressure in the outlet and increased pressure in the inlet. However, the pressure drop in the outlet was less than 11 percent with moderate pore size (FIG. 9).

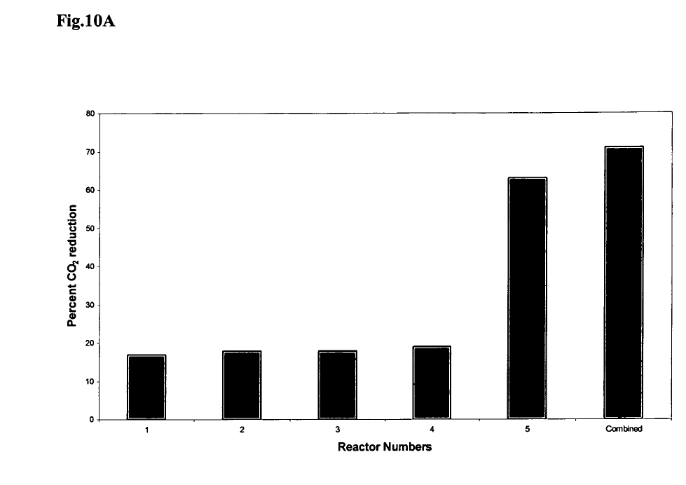

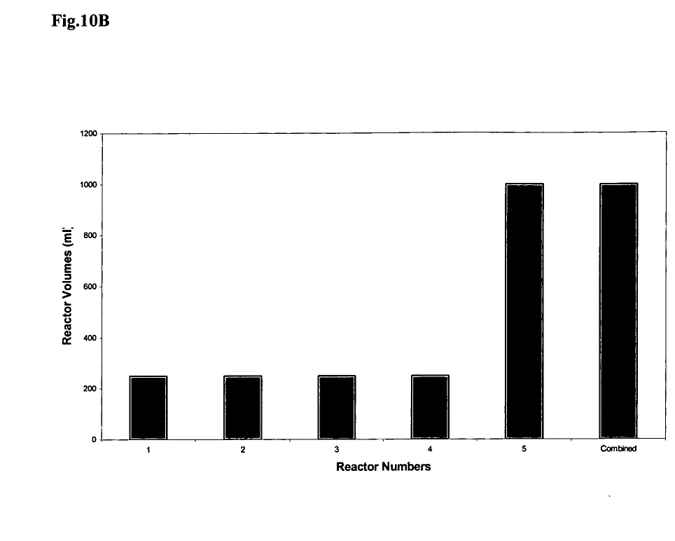

The efficiency of the single versus multiple reactors for CO₂ reduction. The multiple reactors (FIG. 10A) with incremental volume (FIG. 10B) added up to a reactor (FIG. 10A) with equal combined volume (Figure B) were better in reducing the CO₂ from the emission stream than a single reactor with equal combined volume. Using four reactors of 250 ml and a single reactor of 1000 ml it has been found that the multiple reactors provided better extraction/reduction of CO₂ (FIGS. 10A & B). Using this enzymatic reactors it was found that CO₂ could be extracted from emission stream much in the same fashion that solvent extraction is done for organics. Thus using multiple reactors, reduction of carbon dioxide roughly obeys the equation:
Aₘᵣ=A(KV₁/KV₁+V₂)ⁿ
K: distribution coefficient for carbon dioxide; K=C^{gas}/C^{solution}
Aₘᵣ: the amount of CO₂ left in the emission stream after n reactors
A: the amount of CO₂ in the stream without any reactor
V₁: the volume of emission gas used
V₂: the volume of water used for solvation of CO₂ in each reactor
   Experimental Procedures:

Carbonic anhydrase. The carbonic anhydrase from thermophilic Methanobacterium thermoautotrophicum was cloned in pET19b vector using standard molecular biology protocols as described elsewhere (Smith and Ferry, 1999). The cloned enzyme was expressed in E. coli BL21DE3 plysS transformed with a plasmid vector (pET19b) carrying the DNA sequence and purified using Ni—NTA resin column and was used in the reactor core after immobilization. Recombinant human CA isoform IV which was also used in identical studies was purified using E. coli BL21DE3 plysS transformed with a plasmid vector (pET11d) carrying the DNA sequence of human CA IV, kindly provided by Dr. William Sly as research gift. The enzyme was expressed and purified following published protocols (Waheed et. al., 1997). The bovine and human erythrocyte carbonic anhydrase were procured from Sigma Chemical Co., St. Louis, Mo.

Assay of Carbonic anhydrase. Carbonic anhydrase was activity was assayed using an electrometric method (Wilbur and Anderson, 1948). A 50 μl protein solution was diluted to 4 ml of pre-chilled 50 mM HEPES (N-2-hydroxethylpiperazine-N′-ethanesulfonic acid) buffer, pH 8.0. For assay at different pH, 50 mM HEPES was used above pH 7.0 and 50 mM MES (2N-morpholinoethanesulfonic acid) below pH 7.0 were used. The mixture was stirred and maintained on ice for several minutes. The assay was initiated by the addition of 10 ml of ice-cold, CO₂-saturated water into the reaction vessel. The change in pH from 8.0 to 7.0 at 25° C. was monitored using a bench top pH meter and semi-micro combination electrode and the signal was directed to a chart recorder. CA activity is expressed in Wilbur-Anderson (WA) units per mg of protein and was calculated using the formula [(t₀/t−1)×10]/mg protein, where to and t represent the time required for the pH to change from 8.0 to 7.0 in a buffer control and CA sample respectively. A micromethod was also used to determine CA activity for some selected samples (Maren, 1960) to determine whether the activity measured with electrometric method have good correlation.

Immobilization. The carbonic anhydrase was immobilized using different coupling methods on steel matrix coated with glass, polystyrene or silica (Bhattacharya et. al., 2003).

Preparation of the Silanized Carrier. The iron fillings from a lathe machine was collected, 30-45-mesh particles was used for silanization. For immobilization about 10 mg CA in Tris or HEPES buffer pH 8.0 was used for immoblization per gram matrix. The inorganic support material is first treated with organo-functional silane as described elsewhere (Bunting and Laidler, 1972). The silane reacts with available oxide groups on the carrier surface leaving an organic functional group available for coupling to the enzyme. The reaction of the carrier with gamma-aminopropyl-triethoxy-silane was used for coupling. Silane polymerizes across the surface of the carrier anchored at intervals (Bunting and Laidler, 1972; Kobayashi and Moo-Young, 1973). The amino derivative was covalently coupled using carbodiimides as described for other enzymes and matrices before (Chakrabarti et. al., 2003a) or converted into carboxyl derivative using alkylamine-carrier with succinic anhydride using published protocol for other enzymatic entities (Harhen and Barry, 1990).

Preparation of glass coated cyanogens bromide activated carriers. A very thin layer of glass was coated on iron filings (40-60-mesh) and this thin layer of glass (Silicosteel; Restek) was used for direct attachment of carbonic anhydrase using cyanogen bromide mediated coupling (Srinivasan and Bumm, 1974; Chickere, et. al., 2001). About 10 mg CA in HEPES Buffer pH 8.0 was applied per gram of matrix for immobilization.

Determination of mechanical stability of the immobilization matrix. The particle size distribution of controlled pore inert matrices were measured as a function of applied load to a standard volume of materials in a punch and die set within a pressure range 50 to 200 and 200 to 2500 psi (Eaton, 1974; Eaton 1976). Mercury intrusion porosimetry utilizing an Aminco-Winslow Porosimeter (Messing, 1970; Messing, 1974) was used to determine pore density of the immobilized porous materials. For this purpose both BSA and CA II was immobilized using all four methods and operated under pressures 50 to 200 and 200-2500 psi and the average pore diameter was estimated to determine breakage of matrix.

Measurement of Pressure and Carbon dioxide in the emission gas. The pressure of the emission stream in the inlet and outlet was measured using HD8804 K pressure and temperature kit equipped with appropriate pressure probes. Some measurements were also made using Testo 525 instrument (Hotek Technologies, Tacoma Wash.). For carbon dioxide measurement in the emission gas Testo 400 IAQ kit equipped with 0632 1240 and 0635 1240 CO₂ probe was used(Hotek Technologies, Tacoma Wash.).

### REFERENCES.

Alber, B and Ferry, J G (1994) A carbonic anhydrase from the archaeon Methanosarcina thermophila. Proc. Natl. Acad. Sci., USA 91: 6909-6913.
Azari F, Nemat-Gorgani M. (1999) Reversible denaturation of carbonic anhydrase provides a method for its adsorptive immobilization. Biotechnol Bioeng. 62:193-9.
Badger, M R and Price, G D (1994) The role of carbonic anhydrase in photosynthesis. Ann. Rev. Plant Physiol. Plant Mol. Biol. 45: 369-392.
Beckman, E. J. (1999) Making polymers from carbon dioxide. Science 283: 946-947.
Bhattacharya S. K. (2001) Conversion of carbon dioxide from ICE engine exhausts by fixation. U.S. Pat. No. 6,258,335, pp. 1-18.
Bhattacharya, S., Chakrbarti, S and Bhattacharya, S. K. (2002) Bioprocess for recyclable CO₂ fixation: A general description. In Recent Research Developments in Biotechnol. Bioengg. (Bhattacharya, S. K., Chakrabarti, S. and Mal, T. K. eds.) Research Signpost., Kerala, pp. 109-120.
Bhattacharya, S., Schiavone, M., Chakrabarti, S and Bhattacharya, S. K. (2003) CO₂ hydration by immobilized Carbonic anhydrase. Biotechnol. Appl. Biochem. In Press.
Bunting, P. S., Laidler, K. J. (1972) Kinetic studies on solid supported β-galactosidase. Biochemistry 11: 4477-4483.
Chakrabarti S, Bhattacharya, S and Bhattacharya, S. K. (2003a) Immobilization of D-ribulose-1,5-bisphosphate carboxylase/oxygenase: A step toward carbon dioxide fixation bioprocess. Biotechnol. Bioengg. 81: 705-711.
Chakrabarti S, Bhattacharya, S and Bhattacharya, S. K. (2003b) Biochemical Engineering: cues from cells. Trends in Biotechnology. 21: 204-9.
Chikere A C, Galunsky B, Schunemann V V, Kasche V V. (2001) Stability of immobilized soybean lipoxygenases: influence of coupling conditions on the ionization state of the active site Fe. Enzyme Microb Technol. 28 :168-175.
Coleman, J R (1991) The molecular and biochemical analysis of CO₂ concentrating mechanisms in cyanobacteria and green algae. Plant Cell Environ. 14: 861-867.
Eaton, D. L. (1976) The optimization of Porous glasses for immobilized enzyme (IME) systems. Med. Res. Eng. 12: 17-22.
Eaton, D. L. (1974) The optimization of Porous glasses for immobilized enzyme (IME) systems. Adv. Exp. Med. Biol. 42: 241-258.
Gagnon, D. R., Coleman, P L, Ortina, G J, Lyons, C S, Milbrath, D S, Rasmussen, J K, Stahl, J B (1994) Porous supports having azalactone-functional surfaces. U.S. Pat. No. 5,344,701.
Harhen B, Barry F. (1990) Immobilization of proteolytic enzymes. Biochem Soc Trans. 18: 314-5.
Hewett-Emmett, D and Tashian, R E (1996) Functional diversity, conservation, and convergence in the evolution of the α-, β-, and γ-carbonic anhydrase gene families. Mol. Phylogenet. Evol. 5: 50-77.
Holmes, R S (1977) Purification, molecular properties and ontogeny of carbonic anhydrase isozymes. Evidence for A, B and C isozymes in avian and mammalian tissues. Eur. J Biochem. 78: 511-520.
Ingle R K and Coleman, B (1975) Carbonic anhydrase levels in blue-green algae. Can. J Bot. 53: 2385-2387.
Joos, F., Plattner, G.-K., Stocker, T. F., archal, O. and Schmittner, A. (1999) Global warming and marine carbon cycle feedbacks on future atmospheric CO₂. Science. 284: 464-467.
Kaplan, A, Schwarz, R, Ariel, R, Reinhold, L (1990) The CO₂-concentrating mechanism of cyanobacteria: physiological, molecular and theoretical studies. Bot Mag (Tokyo) Special Issue: 53-71.
Karrasch M, Bott, M, Thauer, R K (1989) Carbonic anhydrase activity in acetate grown Methanosarcina barkeri. Arch. Microbiol. 151: 137-142.
Kobayashi, T., Moo-Young, M. (1973) The kinetic and mass transfer behavior of immobilized invertase on ion-exchange resin beads. Biotechnol. Bioengg. 15: 47-67.
Liu, D M, Chen, I-Wei (2001) Encapsulation of biomaterials in porous glass-like matrices prepared via an aqueous colloidal sol-gel process. U.S. Pat. No. 6,303,290.
Manecke G, Schlunsen J. (1976) Immobilization of enzymes on neutral and ionic carriers. Methods Enzymol. 44:107-17.
Manecke G, Vogt H G. (1980) Some new reactive polymers for the immobilization of enzymes. Biochimie. 62: 603-13.
Maren, T. H. (1960) A simplified micromethod for the determination of carbonic anhydrase and its inhibitors. J. Pharmacol. Exp. Ther. 130: 26-29.
Maren, T H (1967) Carbonic anhydrase: chemistry, physiology, and inhibition. Physiol Rev. 47: 595-781.
Maren, T H and Sanyal, G (1983) The activity of sulfonamides and anions against the carbonic anhydrase of animals, plants and bacteria. Ann. Rev. Pharmacol. Toxicol. 23: 439-459.
Messing, R A (1974) Simultaneously immobilized glucose oxidase and catalase in controlled-pore titania. Biotechnol. Bioengg. 16: 897-908.
Messing, R A (1970) Relationship of pore size and surface area to quantity of stabilized enzyme bound to glass. Enzymologia. 39: 12-14.
Miller, A G, Espie, G S, Canvin, D T (1990) Physiological aspects of CO2 and HCO3-transport by cyanobacteria: a review. Can. J Bot. 69: 1151-1160.
Price, G D, Coleman J R, Badger, M R (1992) Association of carbonic anhydrase activity with carboxysomes isolated from the cyanobacterium Synechococcus PCC7942 Plant Physiol. 100: 784-793.
Salley S O, Song J Y, Whittlesey G C, Klein M D. (1992) Thermal, operational, and storage stability of immobilized carbonic anhydrase in membrane lungs. ASAIO J. 38: M684-7.
Schnur, R (2002) The investment forecast. Nature. 415, 483-4.
Simsek-Ege F A, Bond G M, Stringer J. (2002) Matrix molecular weight cut-off for encapsulation of carbonic anhydrase in polyelectrolyte beads. J Biomater Sci Polym Ed. 13:1175-87.
Smith, K S and Ferry, J G (1999) A plant type (beta-class) carbonic anhydrase in the thermophilic methanoarchaeon Methanobacterium thermoautotrophicum. J. Bacteriol. 181: 6247-53.
Srinivasan V R, Bumm M W. (1974) Isolation and immobilization of beta-D-glucosidase from alcaligenes faecalis. Biotechnol Bioeng. 16:1413-8.
Sultemeyer D, Schmidt, C, Fock, H P (1993) Carbonic anhydrases in higher plants and aquatic microorganisms. Physiol. Plant 88: 179-190.
Suzuki E, Shiiraiwas, Y, Miyachi, S. (1994) The cellular and molecular aspects of carbonic anhydrase in photosynthetic microorganisms. Prog. Phycol. Res. 10: 2-54.
Tashian, R E (1989) The carbonic anhydrases: widening perspectives on their evolution, expression and function. Bioessays. 10: 186-192.
Turkova J. (1976) Immobilization of enzymes on hydroxyalkyl methacrylate gels. Methods Enzymol. 44:66-83.
Waheed, A., Pham, T., Won, M., Okuyama, T. and Sly, W. S. (1997) Human carbonic anhydrase IV: In vitro activation and purification of disulfide-bonded enzyme following expression in Escherichia coli. Protein Expression and Purification. 9: 279-287.
Wilbur, K M, Anderson, N G (1948) Electrometric and colorimetric determination of carbonic anhydrase. J. Biol. Chem. 176: 147-154

1. (canceled)

2. (canceled)

3. A Novel trickling spray reactor/device (shall hitherto be code named as module O) where the biotechnological concentration of CO₂ from exhaust stream will occur as an integral component of our modular devices (module A, B and C) as described in U.S. Pat. No. 6,258,335. The component/module O will allow extraction of solubilized carbon dioxide for feeding into the coupled module A for further biochemical fixation on RuBP to form 3PGA and will be put before module A.
